(19) United States
(12) Patent Application Publication
(10) Pub. No.: US 2025/0281568 A1
(43) Pub. Date: Sep. 11, 2025

(54) METHOD FOR TREATING OR PREVENTING DRUG-INDUCED LIVER INJURY WITH PEPTIDE COMPOSITION

(71) Applicants: Tunghai University, Taichung City (TW); Green Abiobiotechnology Co., Ltd., Taichung City (TW)

(72) Inventors: Chang-Chi Hsieh, Taichung City (TW); Zhuo-En Tsai, Taichung City (TW); Yu-Hsuan Lin, Taichung City (TW); Vipul Wayal, Taichung City (TW); Yi-Hsuan Lai, Taichung City (TW)

(21) Appl. No.: 18/732,205

(22) Filed: Jun. 3, 2024

(30) Foreign Application Priority Data
Mar. 6, 2024 (TW) 113108188

(51) Int. Cl. A61K 38/10; A61K 31/145; A61K 35/57; A61K 38/05; A61K 38/08; A61P 1/16; C12N 9/50; C12P 21/06

(52) U.S. Cl. CPC A61K 38/10; A61K 31/145; A61K 35/57; A61K 38/05; A61K 38/08; A61P 1/16; C12N 9/50; C12P 21/06; C12Y 304/21

(57) ABSTRACT

A method for treating or preventing drug-induced liver injury with a peptide composition includes administering an effective amount of the peptide composition to an individual to improve or treat injury or inflammation of the individual's liver, wherein the peptide composition includes peptides represented by SEQ ID No. 1 to SEQ ID No. 14.

### REFERENCE TO AN ELECTRONIC SEQUENCE LISTING

The content of the electronic sequence listing (sequencelist.xml; Size: 13,139 bytes; and Date of Creation: Jun. 3, 2024) is herein incorporated by reference in its entirety.

### BACKGROUND OF THE INVENTION

### 1. Technical Field

The present invention relates to a method for preventing or treating drug-induced liver injury. More particularly, the invention relates to a method of treating or preventing drug-induced liver injury with a peptide composition.

### 2. Description of Related Art

Acetaminophen (APAP) is widely used in drugs for relieving pain and reducing fever. Acetaminophen has only minor side effects on the human body if used as prescribed by a physician, but is highly hepatotoxic or even conducive to liver failure if taken in an overdose or by an individual with liver injury.

As a non-controlled substance, acetaminophen is easily accessible to the general public, and this may explain why overuse of acetaminophen has been a common cause of drug-induced liver injury (DILI).

### BRIEF SUMMARY OF THE INVENTION

The primary objective of the present invention is to provide a method for treating or preventing drug-induced liver injury with a peptide composition. The peptide composition disclosed herein includes a group of small-molecule peptides capable of maintaining the liver function indices, mitigating the inflammatory response of the liver, enhancing the anti-oxidation ability of the liver, and reducing the activity of oxidative stress caused by the reactive oxygen species (ROS) in the liver. Therefore, by administering an effective amount of the peptide composition to an individual, drug-induced liver injury can be effectively treated or prevented.

To achieve the foregoing objective, the present invention discloses a method for treating or preventing drug-induced liver injury with a peptide composition. The method includes administering an effective amount of the peptide composition to an individual to maintain the normal functions of the individual's liver and to reduce or delay liver injury attributable to oxidation or inflammation. The peptide composition includes peptides represented by SEQ ID No. 1 to SEQ ID No. 14.

The individual stated above is an individual at high risk of drug-induced liver injury. For example, the individual does not take drugs according to physicians' prescriptions, has a liver-related disease, or has abnormal liver functions.

Moreover, the drug-induced liver injury is induced by a drug for relieving pain and reducing fever, such as acetaminophen.

In one embodiment of the present invention, the peptide composition includes a peptide group and amino acids. The peptide group is composed of peptides represented by SEQ ID No. 1 to SEQ ID No. 14, and the amino acids include anserine, taurine, and carnosine.

In one embodiment of the present invention, the peptide composition is a nutritional supplement, a complementary therapy product, or a food.

### BRIEF DESCRIPTION OF THE SEVERAL VIEWS OF THE DRAWINGS

FIG. 1 is an LC-MS/MS chromatogram of a chicken meat hydrolysate having a molecular weight of 3-10 kDa.

FIG. 2 is an LC-MS/MS chromatogram of a chicken meat hydrolysate having a molecular weight less than 3 kDa.

FIG. 3 shows the test result of the serum aspartate aminotransferase (AST) concentration of each group of mice.

FIG. 4 shows the test result of the serum alanine aminotransferase (ALT) concentration of each group of mice.

FIG. 5 shows the test result of the serum alkaline phosphatase (ALP) concentration of each group of mice.

FIG. 6 shows the test result of the serum C-reactive protein (CRP) concentration of each group of mice.

FIG. 7 shows the test result of the serum interleukin-6 (IL-6) concentration of each group of mice.

FIG. 8 shows the test result of the serum monocyte chemoattractant protein-1 (MCP-1) concentration of each group of mice.

FIG. 9 shows the test result of the liver superoxidase dismutase (SOD) concentration of each group of mice.

FIG. 10 shows the test result of the liver glutathione peroxidase (GPx) concentration of each group of mice.

FIG. 11 shows the test result of the liver glutathione (GSH) concentration of each group of mice.

FIG. 12 shows the H&E-stained liver section of each group of mice.

FIG. 13 shows the liver section of each group of mice after immunohistochemical staining of 4-hydroxy-2-nonenal (4-HNE).

FIG. 14 shows the liver section of each group of mice after immunohistochemical staining of Kelch-like ECH-associated protein 1 (Keap1).

FIG. 15 shows the liver section of each group of mice after immunohistochemical staining of nuclear factor erythroid 2-related factor 2 (Nrf2).

FIG. 16 shows the liver section of each group of mice after immunohistochemical staining of heme oxygenase 1 (HO-1).

FIG. 17 shows the liver section of each group of mice after immunohistochemical staining of GSH.

### DETAILED DESCRIPTION OF THE INVENTION

The present invention discloses a method for treating or preventing drug-induced liver injury with a peptide composition. That is to say, the administration of an effective amount of the peptide composition disclosed herein to an individual can effectively prevent or delay drug-induced liver injury or a related disease.

The individual stated above may be a healthy individual, an individual who has overdosed on a drug, or an individual at high risk of drug-induced liver injury.

In one embodiment of the present invention, the peptide composition disclosed herein has the activity to, for example, protect liver cells, reduce inflammation, slow down the activation of neutrophils and monocytes, and resist oxidation. When administered to the individual, therefore, the peptide composition disclosed herein can lower the aspartate aminotransferase (AST), alanine aminotransferase (ALT), and alkaline phosphatase (ALP) concentrations of the individual's serum and regulate the liver inflammation index, thereby alleviating drug-induced liver injury and maintaining the normal functions of the liver.

In one embodiment of the present invention, the drug-induced liver injury is induced by a drug for relieving pain and reducing fever, such as acetaminophen.

In one embodiment of the present invention, the main active ingredient of the peptide composition is a group of peptides whose molecular weights are not greater than 10 kDa, and the group of peptides includes peptides represented by SEQ ID No. 1 to SEQ ID No. 14.

The group of peptides may be artificially synthesized or be separated from an extract or a protein hydrolysate, wherein the extract or the protein hydrolysate is prepared by a particular process in order to include peptides represented by SEQ ID No. 1 to SEQ ID No. 14. For example, the group of peptides is derived from a chicken meat hydrolysate, and the chicken meat hydrolysate is obtained by a hydrolysis reaction between chicken meat and a hydrolytic enzyme, wherein the hydrolytic enzyme is an alkaline protease, and wherein the reaction conditions of the hydrolysis reaction include a temperature of about 50-60° C. and a hydrolysis reaction time of 100-150 minutes.

In one embodiment of the present invention, the peptide composition further includes large amounts of amino acids such as anserine, taurine, and carnosine.

Acetaminophen (APAP) is the most commonly and extensively used painkiller in the world thanks to its pain relieving and fever reducing properties, and can be used as a single preparation or in combination with other drugs. Generally, the recommended dosage of acetaminophen for adults is 325-650 mg administered orally every 4-6 hours, with the maximum daily dose being 4 g/day. For patients at high risk of hepatotoxicity, the recommended maximum daily dose is 2 g/day, and for children, the recommended dosage is 10-15 mg/kg every 4-6 hours, with the maximum daily dose being 50-75 mg/kg.

The term “drug-induced liver injury (DILI)” refers to a disease that involves functional damage to, or the provocation of an immune response in, liver cells, wherein the functional damage or provocation results from damage caused to the liver cells by the toxic metabolite(s) produced by the metabolism of a drug. Generally, drugs that may cause drug-induced liver injury include acetaminophen, tetracycline, phenobarbitone, anti-tuberculosis (TB) agents, traditional Chinese medicinal herbs, and so on.

The term “chicken meat hydrolysate” or “chicken meat hydrolysis product” refers to the small-molecule peptides or amino acids derived from chicken meat (protein) by hydrolysis, wherein the hydrolysis includes chemical hydrolysis and enzymatic hydrolysis. Chemical hydrolysis entails a hydrolysis reaction taking place through an acidic or alkaline solution, whereas enzymatic hydrolysis mainly uses an enzyme to hydrolyze a protein.

The technical features of the present invention and their intended effects are detailed below with reference to some experimental examples in conjunction with the accompanying drawings.

### Example 1: Preparation of a Peptide Composition

Chicken meat was added with distilled water having 10 times the weight of the chicken meat, then homogenized, and brought to a boil. After boiling continuously for about 2 hours, the scum was removed, and the broth was taken and then added with 0.2 M NaHPO₄. Once the pH value of the broth was adjusted to 7.5, 1% alcalase was added, and hydrolysis took place at a temperature of about 55° C. After about 2 hours of hydrolysis, a chicken meat hydrolysate solution was obtained. The chicken meat hydrolysate solution was subjected to a bicinchoninic acid assay (BCA) for protein concentration determination and was subsequently concentrated to produce a chicken meat hydrolysate, whose BCA protein concentration was 20 mg/mL.

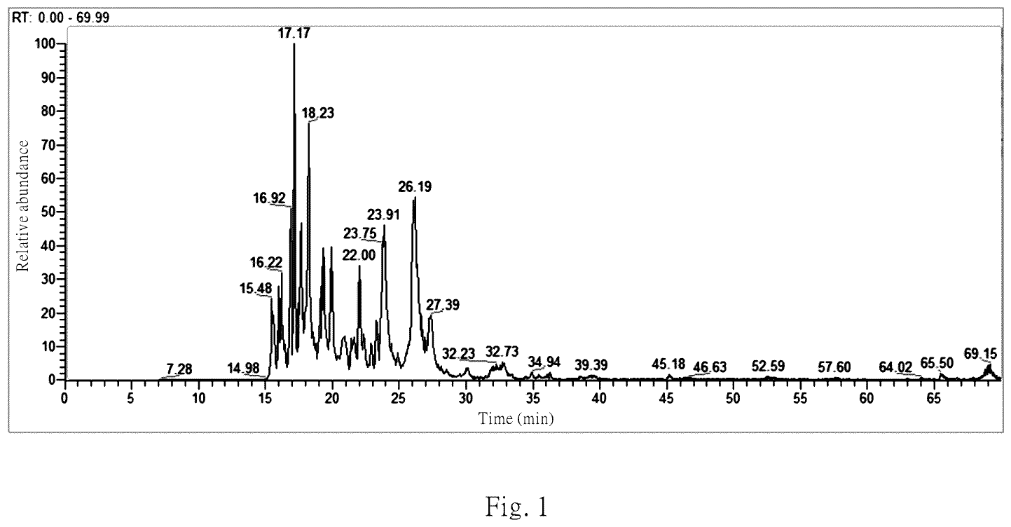

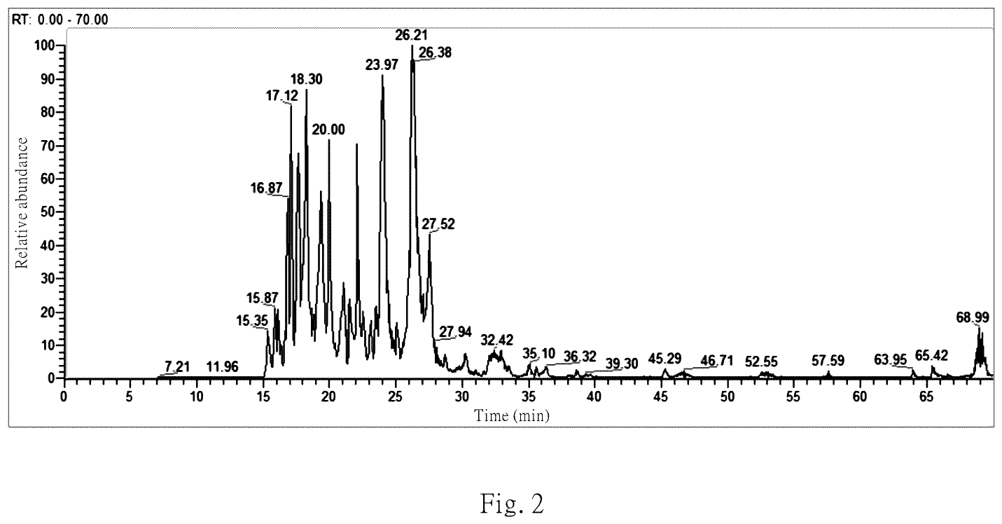

The chicken meat hydrolysate was filtered and centrifuged (at 7500 rpm for about 60 minutes) and then went through fractionation by sequential filtration through membrane filters of different particle size limits such that a hydrolysate having a molecular weight of 3-10 kDa and a hydrolysate having a molecular weight less than 3 kDa were obtained. The two hydrolysates were separately analyzed by liquid chromatography-tandem mass spectrometry (LC-MS/MS), and the analysis results are shown in FIG. 1 and FIG. 2.

The amino acid and peptide constituents of those chicken meat hydrolysate fractions having molecular weights less than 10 kDa were subsequently analyzed, and the analysis results are shown in Tables 1 and 2. It can be known from the results in Table 1 that the chicken meat hydrolysate fractions having molecular weights less than 10 kDa contained anserine, taurine, and carnosine in high concentrations.

TABLE 1
Amino acid constituents of the chicken meat
hydrolysates and their quantification results

| | Molar concentration | Concentration |
|---|---|---|
| Amino acid | (μM) | (mg/L) |
| Anserine | 1476.90 | 354.84 |
| Taurine | 1421.20 | 177.86 |
| Carnosine | 731.54 | 165.50 |
| Threonine | 334.66 | 39.86 |
| Phenylalanine | 206.02 | 33.82 |
| Methionine | 204.75 | 30.74 |
| Proline | 203.63 | 23.44 |
| Lysine | 118.16 | 17.27 |
| Histidine | 117.25 | 15.43 |
| Leucine | 99.45 | 15.38 |
| Tryptophan | 69.98 | 14.29 |
| Tyrosine | 55.27 | 8.16 |
| Isoleucine | 50.93 | 6.68 |
| Valine | 45.06 | 6.47 |
| Glutamine | 35.04 | 5.12 |
| Cystine | 7.98 | 1.61 |
| γ-Aminobutyric acid | 6.69 | 8.23 |
| Homocysteine | 4.94 | 6.68 |

TABLE 2
Analysis of the peptide constituents of the
chicken meat hydrolysates

| SEQ ID No. | Sequence |
|---|---|
| 1 | EPAPPPEE |
| 2 | NGQGGIPGLPGMGPGSS |
| 3 | APKIPEGE |
| 4 | TPPAPEHPPDAE |
| 5 | RIPAPDEPR |
| 6 | EAPAPEPEPEKPK |
| 7 | SPPAVPVHPA |
| 8 | LPIKDPHVD |
| 9 | KPAPPKPEPKPK |
| 10 | APAPAPAPAPAPAPA |
| 11 | PEPPPKEPE |
| 12 | TPPAPEHPPD |
| 13 | TLPIKDPH |
| 14 | TPPNTPDPR |

It can be known from the foregoing results that the chicken meat hydrolysates in this example contained the group of peptides and the amino acids disclosed herein. Therefore, the chicken meat hydrolysates obtained in this example were used in the following examples as the peptide composition of the present invention and had their effects verified.

### Example 2: Animal Test

7-week-old male BALB/cByJNarl mice were randomly grouped, and each group received one of the following treatments for 7 days:
Blank group: Without any treatment;
Control group: With sterilized distilled water orally administered on a daily basis at 10 mL/kg/day;
Low-dosage chicken meat hydrolysate (LCH) group: With the chicken meat hydrolysates orally administered on a daily basis at 50 mg/kg/day;
High-dosage chicken meat hydrolysate (HCH) group: With the chicken meat hydrolysates orally administered on a daily basis at 200 mg/kg/day; and
Carnosine (CAR) group: With carnosine orally administered on a daily basis at 50 mg/kg/day.

After being raised under the corresponding conditions for 7 days, each group of mice was weighed and then fasted for 12 hours. After fasting, each group of mice was intraperitoneally injected with an acetaminophen solution at 300 mg/kg. 24 hours after the injection, the mice were sacrificed, and blood was collected from the mice.

The body weight measurements show no significant difference in body weight among the groups of mice, and this indicates that the peptide composition disclosed herein had no negative effect on the health of the mice, regardless of whether the dosage was low or high.

After the mice were sacrificed, the organs of each group of mice were weighed, and it was found that there was no significant difference in the weight of heart or the weight of liver among the groups of mice, that the liver weight of the control group of mice was significantly higher than those of the other groups of mice, and that the liver weight of the LCH group of mice was significantly higher than those of the blank group, the HCH group, and the CAR group.

### Example 3: Biochemical Analysis of Blood

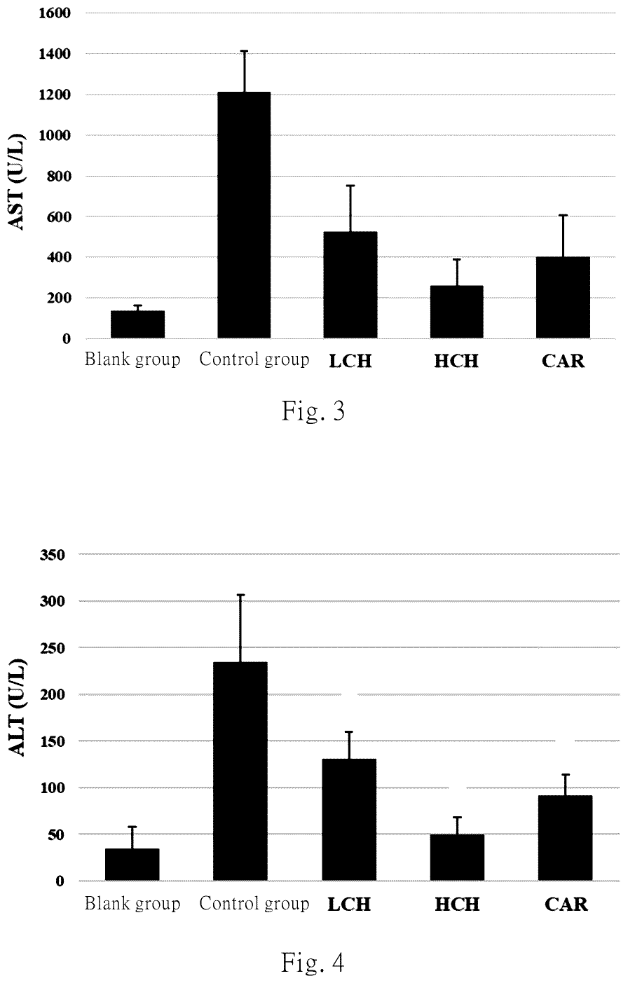

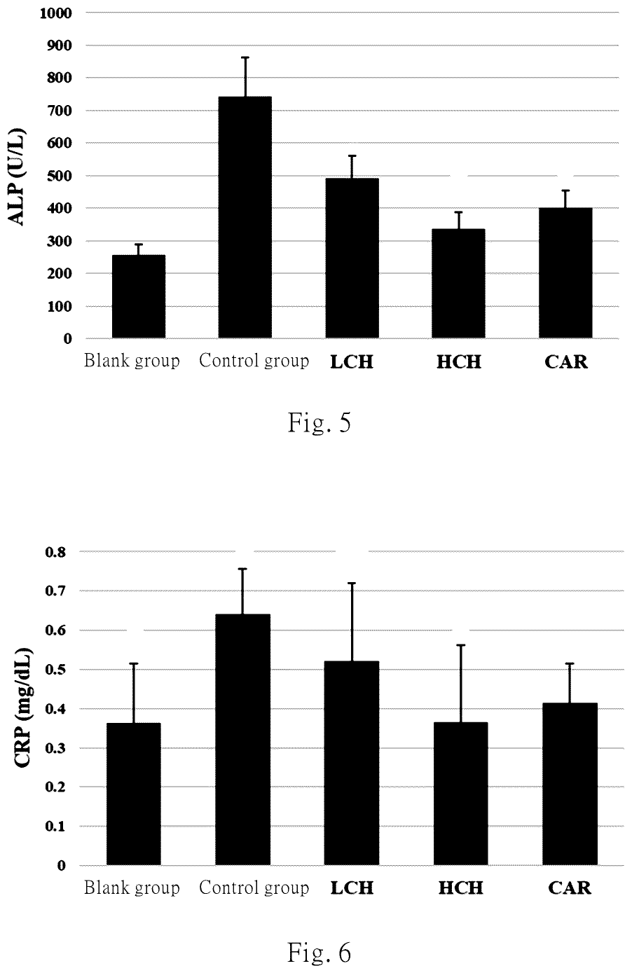

The blood collected from each group of mice in example 2 was analyzed with a commercial assay kit in order to determine the concentrations of aspartate aminotransferase (AST), alanine aminotransferase (ALT), alkaline phosphatase (ALP), C-reactive protein (CRP), interleukin-6 (IL-6), and monocyte chemoattractant protein-1 (MCP-1) in the serum of each group of mice. The analysis results are shown in FIG. 3 to FIG. 8.

According to the results in FIG. 3 to FIG. 5, the serum AST, ALT, and ALP concentrations of the LCH group, the HCH group, and the CAR group of mice were significantly lower than those of the control group, and the serum AST and ALT concentrations of the HCH group of mice were not significantly different from those of the blank group.

According to the results in FIG. 6, the serum CRP concentrations of the HCH group and the CAR group of mice were significantly lower than that of the control group, and the serum CRP concentrations of the HCH group and the CAR group of mice were not significantly different from that of the blank group.

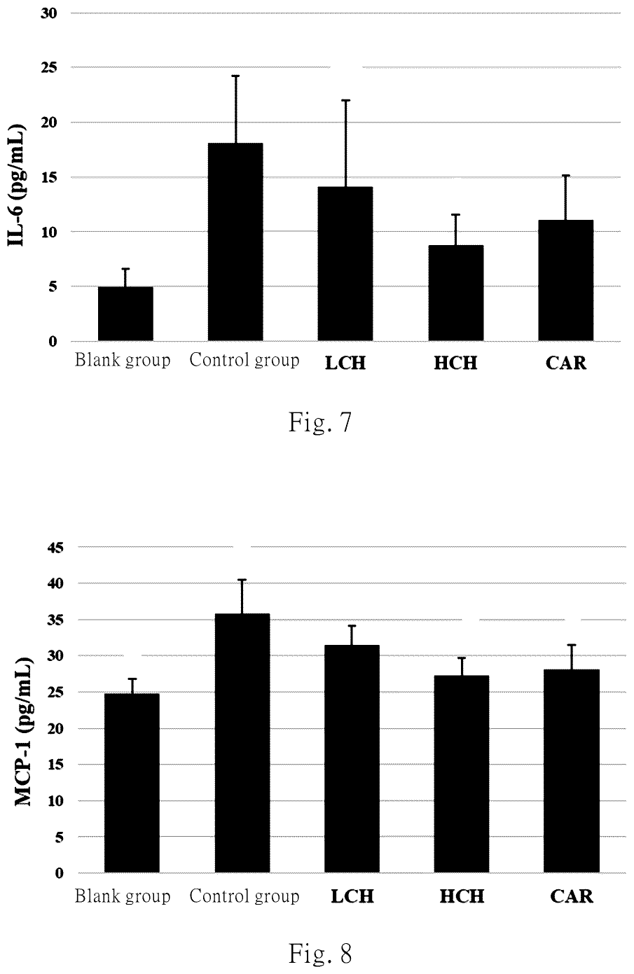

According to the results in FIG. 7, the serum IL-6 concentrations of the HCH group and the CAR group of mice were lower than that of the control group. According to the results in FIG. 8, the serum MCP-1 concentrations of the LCH group, the HCH group, and the CAR group of mice were significantly lower than that of the control group.

It can be inferred from the results in FIG. 3 to FIG. 8 that the peptide composition disclosed herein is indeed capable of and effective in protecting liver cells from drug-induced liver injury or liver inflammation and can therefore be used to prevent or treat drug-induced liver injury.

### Example 4: Analysis of Proteins in the Liver

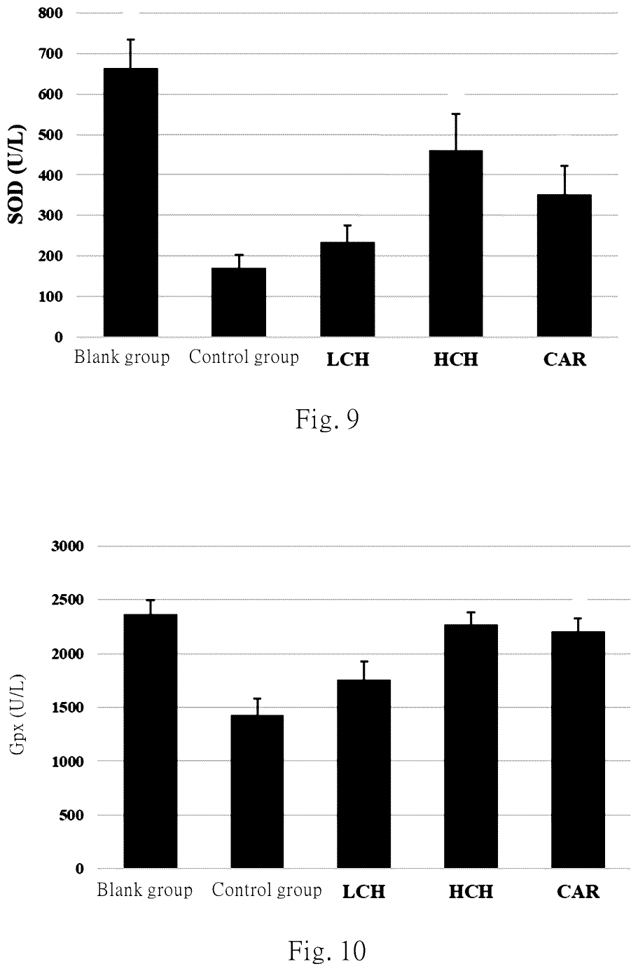

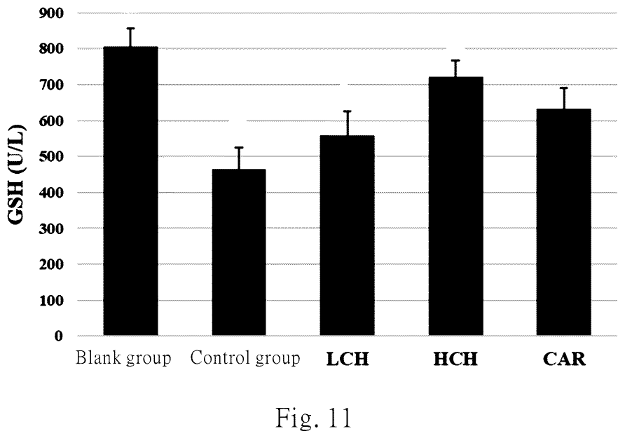

Livers were collected from each group of mice in example 2 and were analyzed in order to determine the concentrations of superoxidase dismutase (SOD), glutathione peroxidase (GPx), and glutathione (GSH) in the livers. The analysis results are shown in FIG. 9 to FIG. 11.

It can be known from the results in FIG. 9 to FIG. 11 that the liver SOD, GPx, and GSH concentrations of the LCH group, the HCH group, and the CAR group of mice were significantly higher than those of the control group of mice. This proves that the peptide composition disclosed herein has the activity to resist the generation of free radicals and to maintain the balance between oxidation and reduction in an individual's body and can therefore protect liver cells from injury or a loss of functions. In addition, by preventing the GSH in the liver from being depleted by interaction with acetaminophen, the peptide composition disclosed herein can increase or maintain the GSH concentration in the liver to prevent or delay drug-induced liver injury.

### Example 5: Analysis of Stained Liver Tissue Sections

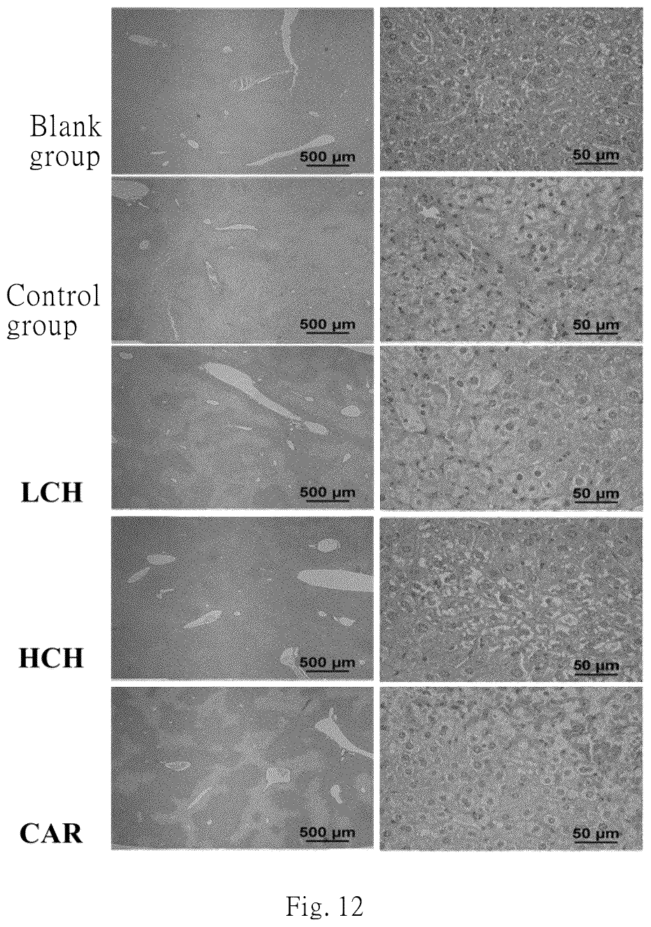

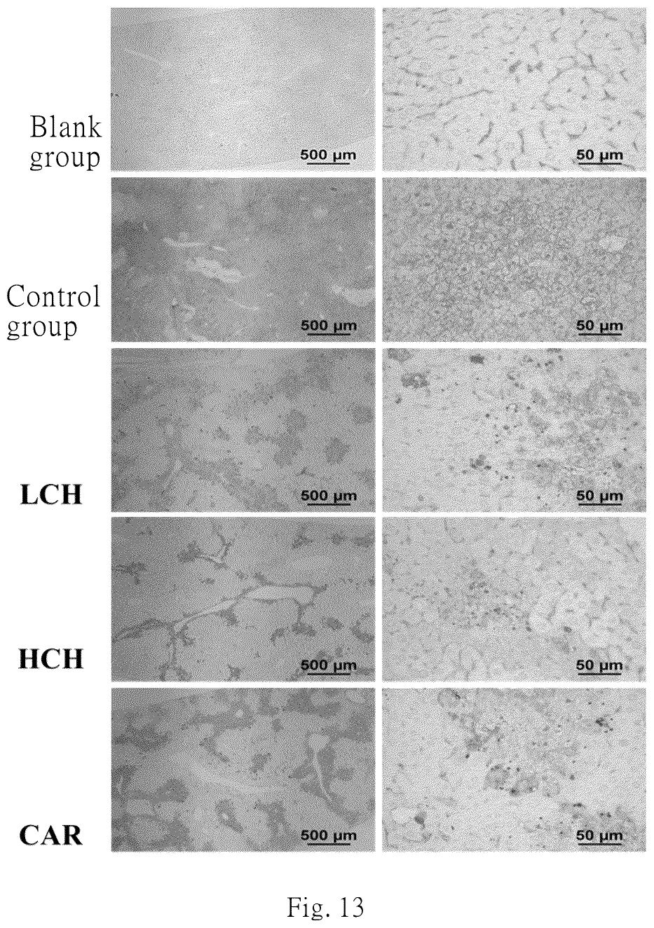

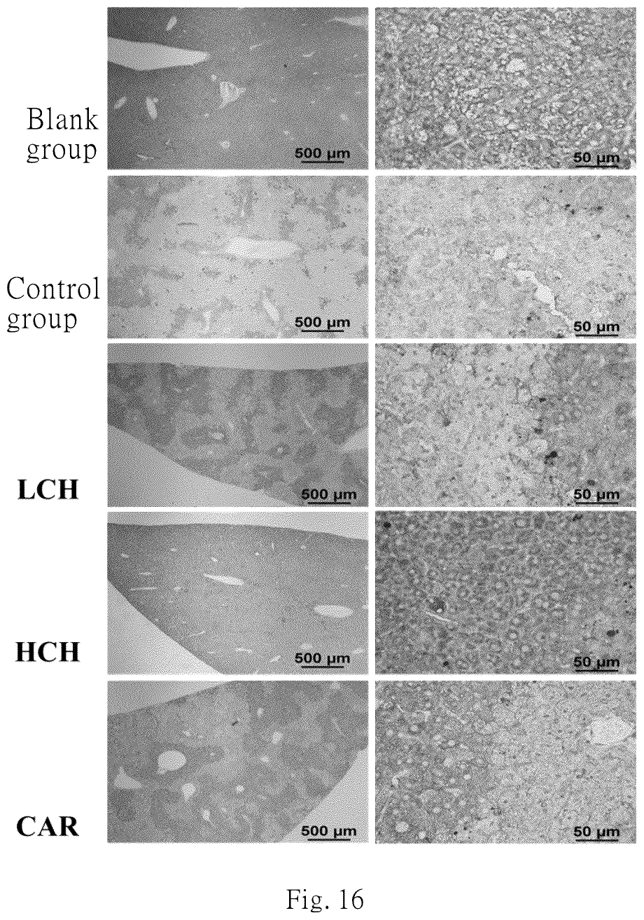

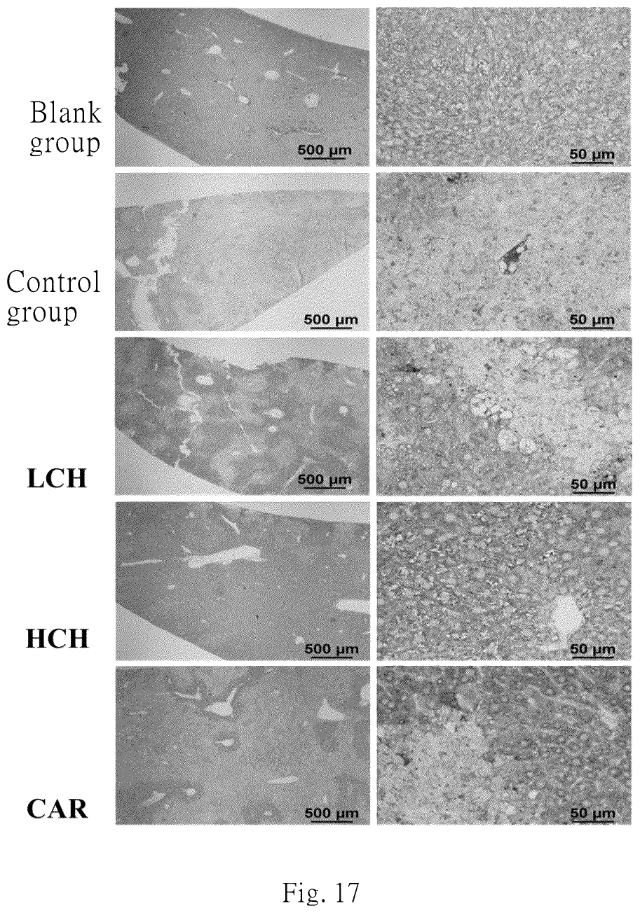

The liver tissue collected from each group of mice in example 2 was sectioned and then subjected to immunohistochemical staining with hematoxylin and eosin (H&E) and with different antibodies. The results are shown in FIG. 12 to FIG. 17.

It can be known from the results in FIG. 12 that the liver cells of the control group, the LCH group, and the CAR group of mice were significantly damaged in comparison with those of the blank group, that the liver cells of the LCH group and the CAR group of mice were damaged to a smaller extent than those of the control group, and that the liver cells of the HCH group of mice had no significant damage in comparison with those of the other groups. This indicates that the peptide composition disclosed herein can protect liver cells, slow down liver injury, and thereby prevent or treat drug-induced liver injury or other related diseases.

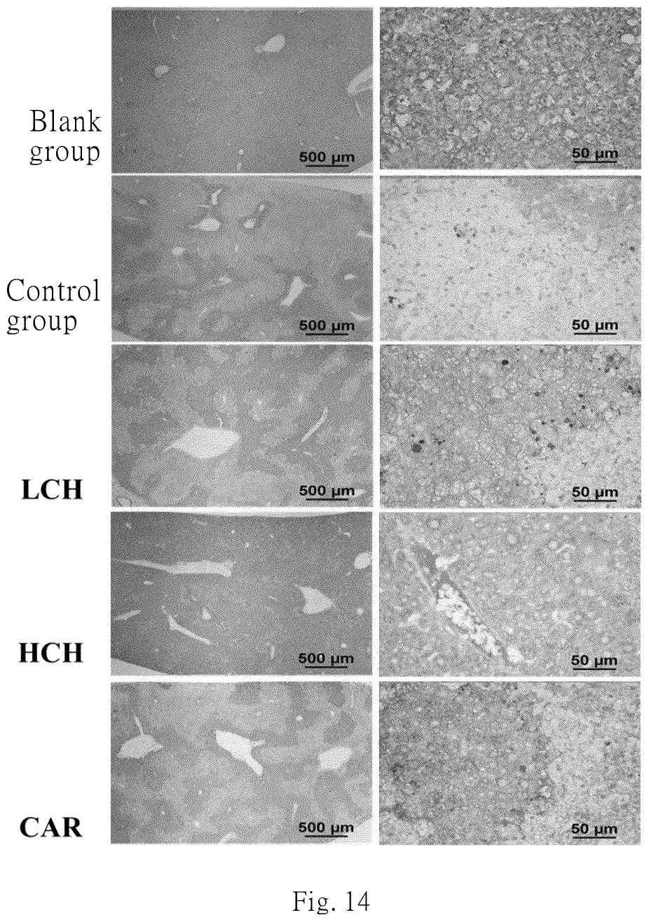

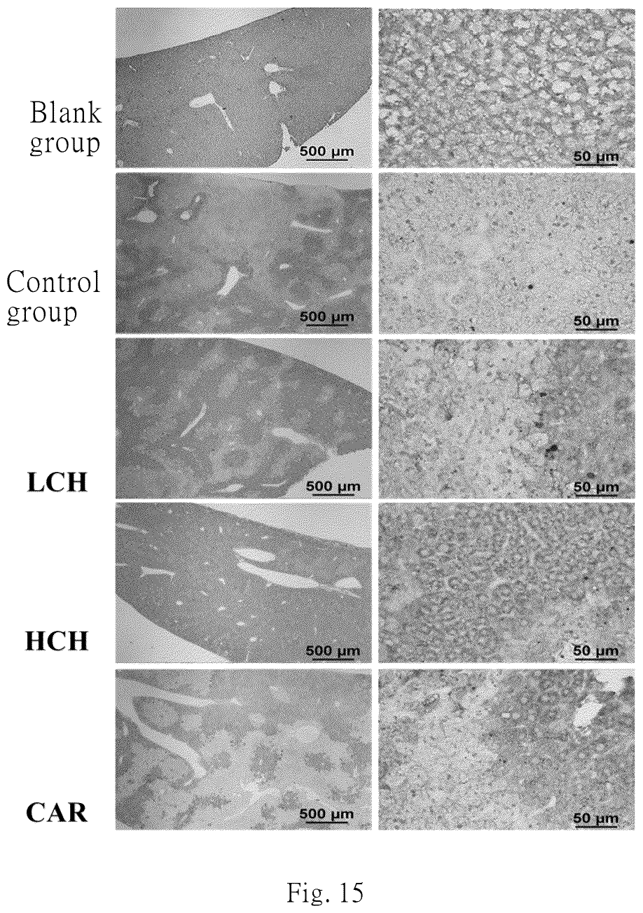

It can be known from the results in FIG. 13 and FIG. 14 that the expression amounts of 4-hydroxy-2-nonenal (4-HNE) and of Kelch-like ECH-associated protein 1 (Keap1) were significantly less in the livers of the LCH group, the HCH group, and the CAR group of mice than in those of the control group. It can also be known from the results in FIG. 15 to FIG. 17 that the expression amounts of nuclear factor erythroid 2-related factor 2 (Nrf2), of heme oxygenase 1 (HO-1), and of GSH were significantly greater in the livers of the LCH group, the HCH group, and the CAR group of mice than in those of the control group. The immunohistochemical staining results prove again that the peptide composition disclosed herein has high anti-oxidation activity and is therefore capable of and effective in protecting liver cells from injury or a loss of functions.

What is claimed is:

1. A method for treating or preventing drug-induced liver injury with a peptide composition, comprising: administering an effective amount of the peptide composition to an individual to improve or treat injury or inflammation of the individual's liver, wherein the peptide composition comprises peptides of SEQ ID No. 1 to SEQ ID No. 14.

2. The method for treating or preventing drug-induced liver injury with a peptide composition as claimed in claim 1, wherein the peptide composition further comprises amino acids, and the amino acids are anserine, taurine, and carnosine.

3. The method for treating or preventing drug-induced liver injury with a peptide composition as claimed in claim 1, wherein the drug-induced liver injury is induced by a drug for relieving pain and reducing fever.

4. The method for treating or preventing drug-induced liver injury with a peptide composition as claimed in claim 3, wherein the drug for relieving pain and reducing fever is acetaminophen.

5. The method for treating or preventing drug-induced liver injury with a peptide composition as claimed in claim 1, wherein the peptide composition is a nutritional supplement, a complementary therapy product, or a food.

6. The method for treating or preventing drug-induced liver injury with a peptide composition as claimed in claim 1, wherein the peptide composition is a protein hydrolysate.

7. The method for treating or preventing drug-induced liver injury with a peptide composition as claimed in claim 1, wherein the peptides of SEQ ID No. 1 to SEQ ID No. 14 are derived from a chicken meat hydrolysate having a molecular weight less than 10 kDa.

8. The method for treating or preventing drug-induced liver injury with a peptide composition as claimed in claim 7, wherein the chicken meat hydrolysate is obtained by a hydrolysis reaction between chicken meat and a hydrolytic enzyme, the hydrolytic enzyme is an alkaline protease, and reaction conditions of the hydrolysis reaction comprise a temperature of about 50-60° C. and a hydrolysis reaction time of 100-150 minutes.


SEQUENCE LISTING (14 sequences)

SEQ ID NO: 1 - protein, 8 aa, Gallus sp.
EPAPPPEE

SEQ ID NO: 2 - protein, 17 aa, Gallus sp.
NGQGGIPGLPGMGPGSS

SEQ ID NO: 3 - protein, 8 aa, Gallus sp.
APKIPEGE

SEQ ID NO: 4 - protein, 12 aa, Gallus sp.
TPPAPEHPPDAE

SEQ ID NO: 5 - protein, 9 aa, Gallus sp.
RIPAPDEPR

SEQ ID NO: 6 - protein, 13 aa, Gallus sp.
EAPAPEPEPEKPK

SEQ ID NO: 7 - protein, 10 aa, Gallus sp.
SPPAVPVHPA

SEQ ID NO: 8 - protein, 9 aa, Gallus sp.
LPIKDPHVD

SEQ ID NO: 9 - protein, 12 aa, Gallus sp.
KPAPPKPEPKPK

SEQ ID NO: 10 - protein, 15 aa, Gallus sp.
APAPAPAPAPAPAPA

SEQ ID NO: 11 - protein, 9 aa, Gallus sp.
PEPPPKEPE

SEQ ID NO: 12 - protein, 10 aa, Gallus sp.
TPPAPEHPPD

SEQ ID NO: 13 - protein, 8 aa, Gallus sp.
TLPIKDPH

SEQ ID NO: 14 - protein, 9 aa, Gallus sp.
TPPNTPDPR